(19) United States
(12) Patent Application Publication
(10) Pub. No.: US 2004/0044355 A1
(43) Pub. Date: Mar. 4, 2004

(54) Minimally invasive corneal surgical procedure for the treatment of hyperopia

(76) Inventor: Herbert J. Nevyas, Bala Cynwyd, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/231,798

(22) Filed: Aug. 28, 2002

(51) Int. Cl. A61F 9/00

(52) U.S. Cl. 606/166

(57) ABSTRACT

A method of treating hyperopia by using a minimally invasive corneal surgical procedure. A femtosecond laser is used to make a cylindrical cut into the stromal bed of the cornea between the endothelial (inner) cornea and the superficial (outer) cornea, while leaving enough deep and superficial stromal layers to maintain the integrity of the cornea. This allows relaxation forward of the corneal stroma, causing the cornea to bow and the hyperopia to be corrected.

### BACKGROUND OF THE INVENTION

There are presently several common corneal surgical procedures used to treat farsightedness or hyperopia, which occurs when light entering the eye focuses behind the retina, instead of directly on it. Hyperopia is caused by a cornea that is flatter, or an eye that is shorter, than a normal eye. Farsighted people usually have trouble seeing up close, but may also have difficulty seeing far away as well.

Hyperopia of the cornea of a patient's eye has been surgically corrected by making a cylindrical incision into the cornea after a corneal hinged LASIK (Laser Assisted In-situ Keratomileusis) flap having a predetermined thickness is made on the cornea. The cylindrical incision is made into the cornea below the corneal hinged flap and then the cornea flap is closed to complete the procedure. The downside to such a procedure is the possibility of an increase in glare caused by the incision, and the risk of complications related to the healing of the LASIK flap after it is replaced. Such complications may include improper alignment of the LASIK flap, infection, and the perforation of the inner surface of the cornea into the anterior chamber of the eye.

The treatment for hyperopia depends upon several factors such as the patient's age, activities, and occupation. Refractive surgery is an option for adults who wish to see clearly without glasses. A corneal surgical procedure which treats hyperopia while eliminating the need to make a LASIK flap would be highly desirable, so as to minimize the risk of infection and the length of the patient's recovery period.

### BRIEF SUMMARY OF THE INVENTION

The present invention uses a surgical technique whereby a cylindrical cut is made into the stromal bed of the cornea, using femtosecond laser, without the need to make a LASIK flap. Thus, the superficial (external) corneal stroma is not penetrated.

The present invention is a method of surgically treating hyperopia of the cornea of a patient's eye by making a cylindrical incision around the optical axis of the cornea with a computer controlled femtosecond laser. The cornea has an inner surface and an outer surface. The cylindrical incision is created between the inner surface and outer surface of the cornea without disrupting the inner or outer surface of the cornea. The femtosecond laser is centered on the optical axis of the cornea. The diameter of the cylinder having a circumference centered around the optical axis and being located between the outer and inner surfaces of the cornea is inputted into a computer that controls the laser. The diameter is generally inversely proportional to the degree of hyperopia to be corrected. The thickness of the cornea is also inputted into a computer. Based on the thickness of the cornea, the computer calculates the height of the cylinder. Based on the thickness of the cornea and the height of the cylinder, the computer calculates the desired depth of the cylinder below the outer surface of the cornea and above the inner surface of the cornea. The femtosecond laser vertically cuts the circumference of the cylinder into the cornea around the optical axis.

The cylinder may have two ends, a first one of the ends being approximately 120 microns from the outer surface of the cornea and the other one of the ends being approximately 100 microns from the inner surface of the cornea.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows an outline of a patient's eye requiring treatment for hyperopia with an outline of where a cylindrical incision is to be made;

FIG. 2 shows the perspective view of a patient's cornea after a cylindrically shaped incision is vertically ablated therein using a femtosecond laser in accordance with the present invention;

FIG. 3 shows a top plan view of the cylindrical incision of FIG. 2 centered on the optical axis of the cornea; and

FIG. 4 is a flow chart showing method steps used to surgically treat hyperopia of the cornea using a femtosecond laser, in accordance with the present invention.

### DETAILED DESCRIPTION OF THE INVENTION

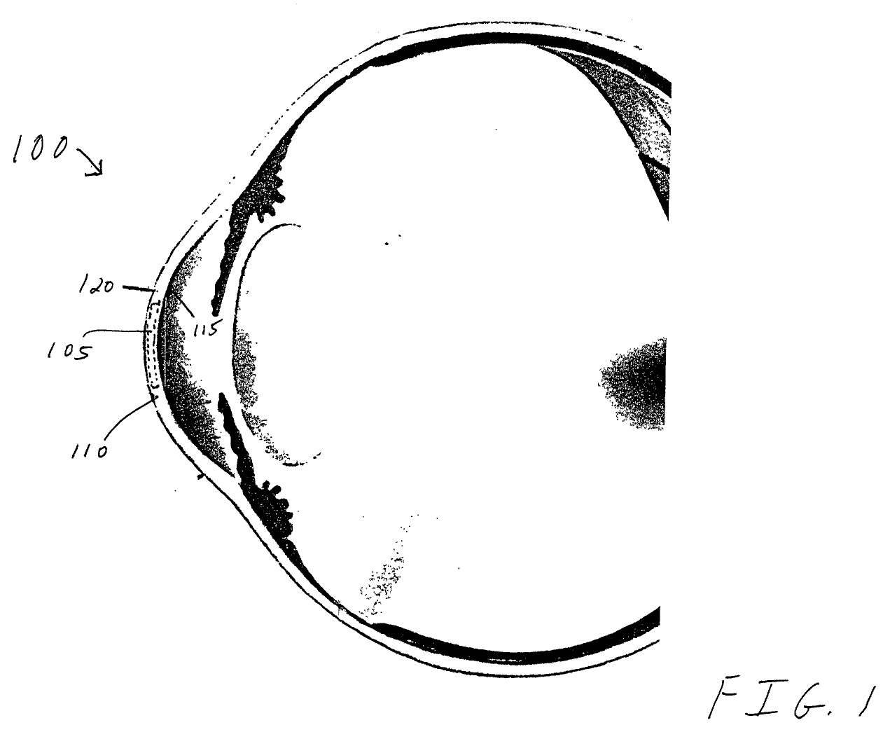

The present invention is a method of treating hyperopia of a patient's eye using a minimally invasive corneal surgical procedure. FIG. 1 shows a cylindrical incision 105 made into the corneal stromal bed 110 of the patient's eye 100 between the endothelial (inner) surface 115 of the cornea and the superficial (outer) cornea 120, while leaving enough deep and superficial stromal layers to maintain the integrity of the cornea. Approximately one hundred microns of undisturbed corneal stroma is left at the endothelial surface 115 of the cornea, and approximately one hundred and twenty microns at the superficial cornea 120. This allows relaxation forward of the corneal stroma, causing the cornea to bow out. A femtosecond laser is used to make the centered cylindrical incision in the stroma, so as to avoid problems associated with creating a LASIK flap and with making a cylindrical incision using a trephine and/or blade.

In accordance with the present invention, a method of surgically treating hyperopia of the cornea of a patient's eye is performed by making a cylindrical incision around the optical axis of the cornea with a computer controlled femtosecond laser. The cornea has an inner surface and an outer surface between which the cylindrical incision is created without disrupting the outer or inner surface of the cornea. The femtosecond laser is centered on the optical axis of the cornea (e.g., the center of the pupil). Various parameters are inputted into the computer that controls the laser. The computer includes a processor on which a laser control program runs. One of the inputted parameters is the diameter of a cylinder having a circumference. The diameter of the cylinder is generally inversely proportional to the degree of hyperopia to be corrected. The thickness of the cornea is also inputted into the computer. Based on the thickness of the cornea, the computer calculates the desired height of the cylinder. Based on the thickness of the cornea and the height of the cylinder, the computer calculates the desired depth of the cylinder below the outer surface of the cornea and above the inner surface of the cornea. The femtosecond laser, under the guidance of the computer program, vertically cuts the circumference of the cylinder into the cornea, the circumference being centered around the optical axis and located between the outer and inner surfaces of the cornea.

The cylinder has two ends, a first one of the ends being approximately 120 microns from the outer surface of the cornea and the other one of the ends being approximately 100 microns from the inner surface of the cornea.

The amount of increased convexity of the cornea that results from the present invention is generally inversely proportional to the diameter of the cylindrical ring of disrupted stromal tissue. For example, a 6 mm diameter incision corrects approximately one diopter of hyperopia. A 4 mm diameter incision corrects approximately four diopters of hyperopia. The increased convexity of the cornea and its corresponding improved optical effect is immediate, after the stromal tissue is cut, and remains permanent after the cut heals. Some changes may occur during the early stages of healing.

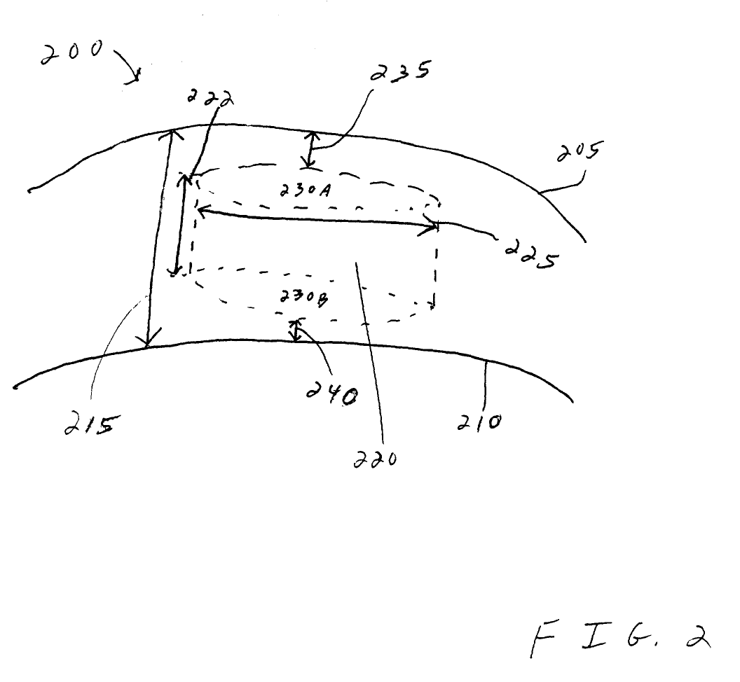

FIG. 2 shows the perspective view of the patient's cornea 200 having an outer surface 205, an inner surface 210 and a thickness 215. A cylindrical incision 220 is made using the femtosecond laser in accordance with the present invention. Cylindrical incision 220 has a height 222, a diameter 225 and two ends 230A, 230B. A first one of the ends 230A is a first predetermined distance 235 below the outer surface 205 of the cornea 200. The other one of the ends 230B is a second predetermined distance 240 above the inner surface 210 of the cornea 200.

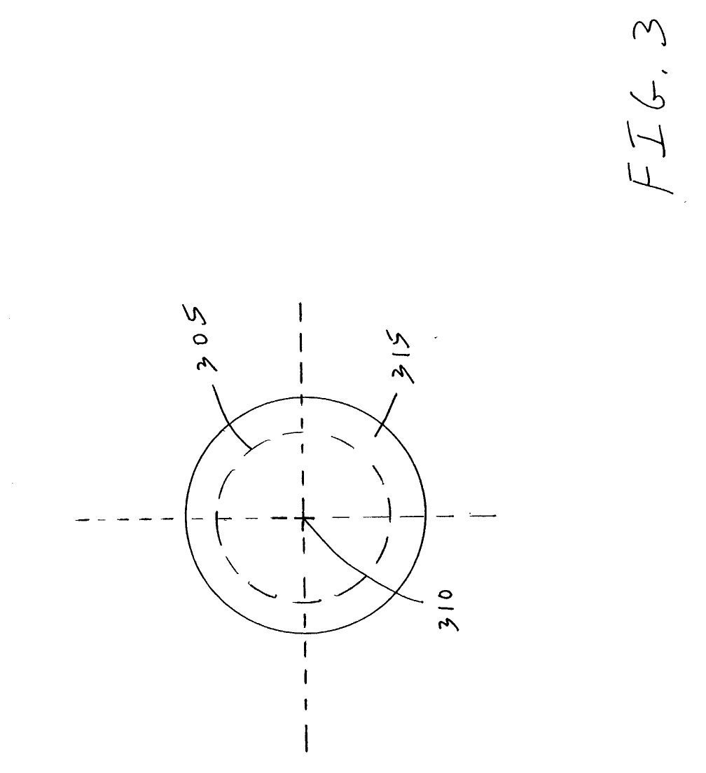

FIG. 3 shows a top plan view of the cylindrical cut 305 of FIG. 2 centered on the optical axis 310 of the cornea 315.

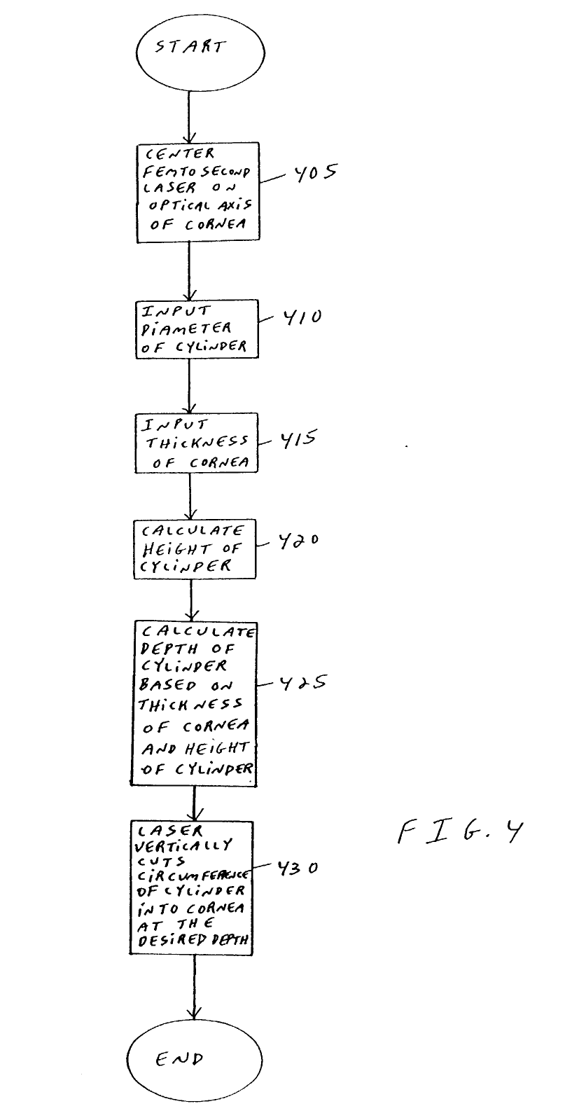

FIG. 4 shows a flow chart which includes method steps used to execute the present invention, whereby hyperopia of the cornea of a patient's eye is surgically treated by making a cylindrical incision with a femtosecond laser. The cornea has an inner surface and an outer surface. In step 405, the femtosecond laser is centered on the optical axis of the cornea (e.g., the center of the pupil). Various parameters are inputted into the computer that controls the laser. The computer includes a processor on which a laser control program runs. One of the inputted parameters is the diameter of a cylinder having a circumference (step 410). The diameter of the cylinder is generally inversely proportional to the degree of hyperopia to be corrected. The thickness of the cornea is also inputted into the computer (step 415). Based on the thickness of the cornea, the height of the cylinder is calculated by the computer (step 420). Based on the thickness of the cornea and the height of the cylinder, the computer calculates the desired depth of the cylinder below the outer surface of the cornea and above the inner surface of the cornea (step 425). In step 430, the femtosecond laser vertically cuts the circumference of the cylinder into the cornea at the desired depth. The circumference of the cylinder is centered around the optical axis and located between the outer and inner surfaces of the cornea.

The cylinder has two ends, a first one of the ends being approximately 100 microns from the outer surface of the cornea and the other one of the ends being approximately 120 microns from the inner surface of the cornea.

The femtosecond laser is programmed to create a cylindrical ablation in the cornea which would allow a controlled ectasia of the cornea to increase the corneal surface convexity and thus treat hyperopia. The inputted parameters, including the diameter of the cut and the thickness of the cornea, are based on the results of a prior examination of the patient's eye. A nomogram may be referred to by the patient's doctor to determine the diameter of the cylinder, based on the results of the examination. An instrument such as an optical or ultrasonic pachymeter may be used by the doctor to determine the thickness of the cornea. The laser control program used to execute the cylindrical cut may include an algorithm that insures that there is adequate remaining corneal stroma on both the inner and outer surfaces of the cornea to maintain the integrity of the cornea after the central stroma of the cornea is cut to treat hyperopia.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention.

What is claimed is:

1. A method of surgically treating hyperopia of the cornea of a patient's eye by making a cylindrical incision around the optical axis of the cornea with a computer controlled femtosecond laser, the cornea having an inner surface and an outer surface, the cylindrical incision being created between the inner and outer surface of the cornea without disrupting the surfaces, the method comprising:
(a) centering the femtosecond laser on the optical axis of the cornea;
(b) inputting, into the computer that controls the laser, the diameter of a cylinder having a circumference, the diameter being generally inversely proportional to the degree of hyperopia to be corrected;
(c) inputting into the computer the thickness of the cornea;
(d) based on the thickness of the cornea, the computer calculating the height of the cylinder;
(e) based on the thickness of the cornea and the height of the cylinder, the computer calculating the desired depth of the cylinder below the outer surface of the cornea and above the inner surface of the cornea; and
(f) the femtosecond laser vertically cutting the circumference of the cylinder into the cornea, the circumference of the cylinder being centered around the optical axis and located between the outer surface and inner surface of the cornea.

2. The method of claim 1 wherein the cylinder has two ends, a first one of the ends being approximately 120 microns below the outer surface of the cornea and the other one of the ends being approximately 100 microns above the inner surface of the cornea.

3. A method of making a cylindrical incision in the cornea of a patient's eye with a computer controlled femtosecond laser to treat hyperopia of the cornea, the method comprising vertically cutting the circumference of a cylinder into the cornea below the outer surface of the cornea, the diameter of the cylinder being generally inversely proportional to the degree of hyperopia to be corrected, wherein the height of the cylinder is determined based on the thickness of the cornea, and the depth of the cylinder below the outer surface of the cornea is determined based on the thickness of the cornea and the height of the cylinder.

4. The method of claim 3 wherein the cylindrical cut is centered around the optical axis of the cornea.

5. The method of claim 3 wherein the cylinder has two ends, a first one of the ends being approximately 120 microns below the outer surface of the cornea and the other one of the ends being approximately 100 microns above the inner surface of the cornea.
